(19) United States
(12) Plant Patent Application Publication
(10) Pub. No.: US 2013/0291264 P1
(43) Pub. Date: Oct. 31, 2013

(54) Petunia

(50) ;

(71) Applicant: Jeffrey R. Fredette, Otisville, MI (US)

(72) Inventor: Jeffrey R. Fredette, Otisville, MI (US)

(21) Appl. No.: 13/506,541

(22) Filed: Apr. 26, 2012

(51) Int. Cl. A01H 5/00

(52) U.S. Cl. PLT/356.23

(57) ABSTRACT

A new and distinct variety of petunia plant as characterized by its distinctive flower color and its dark green foliage.

### LATIN NAME

Petunia

### VARIETAL DENOMINATION

‘DaSaJa1’

### FIELD OF THE INVENTION

The invention is directed to a new variety of a petunia plant and more particularly to a petunia plant that produces a flower of a color different than present petunia plants.

### BACKGROUND OF THE INVENTION

The invention herein is a new and distinct variety of petunia plants. I discovered this plant in a cultivated area on my farm in Otisville, Mich., in March 2010.

This petunia began to exhibit qualities distinctly different from other types of petunias. The color of the flower was quite different than other petunia plants.

I performed asexual reproduction of my new petunia plant by vegetative cuttings on or about and several times since May 2010 at approximately 60 day to 90 day intervals in Otisville Mich.

I have chosen “DaSaJa1” as the varietal name for my new petunia plant.

### SUMMARY OF THE INVENTION

DaSaJa1 is a distinctive, new variety of a petunia plant characterized by its flower color of red to pink shade color streaking from the top of the throat to the petal margin and also by its dark green foliage and the short length of its internodes. These traits are maintained when propagated asexually.

### BRIEF DESCRIPTION OF THE DRAWINGS

FIGS. 1 and 2 are close up photographs of the flowers and the foliage of the petunia plant of the present invention; and

FIGS. 3 and 4 show the overall appearance of the petunia plant of the present invention.

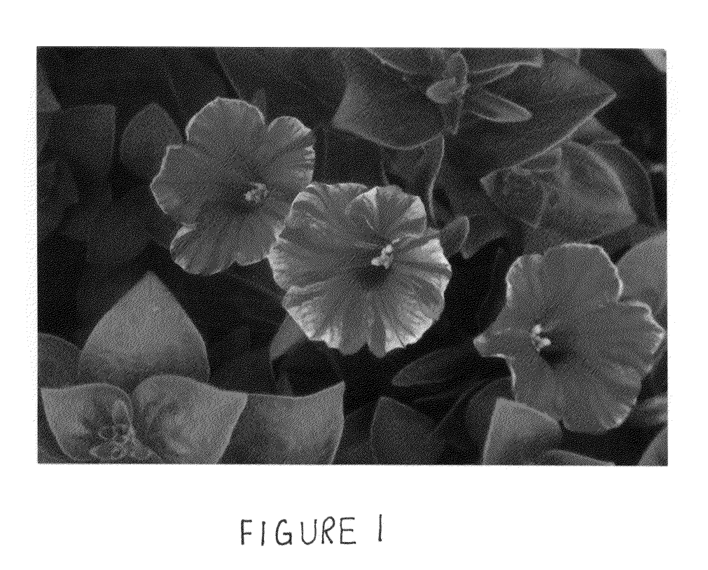

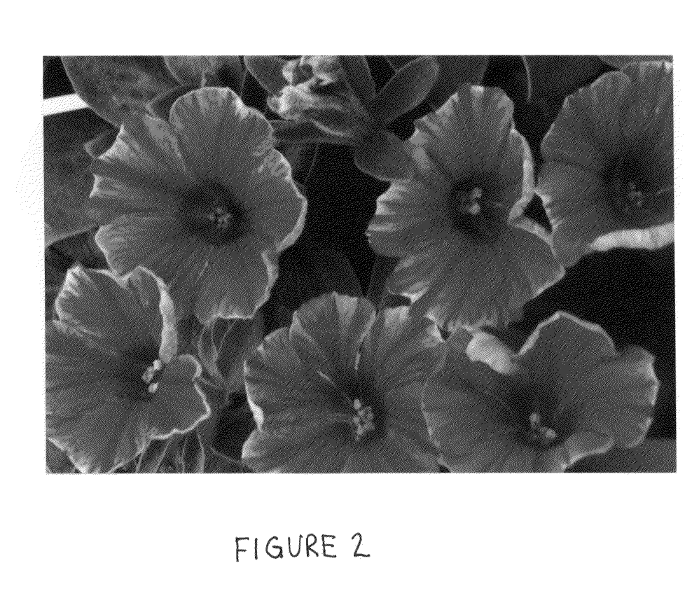

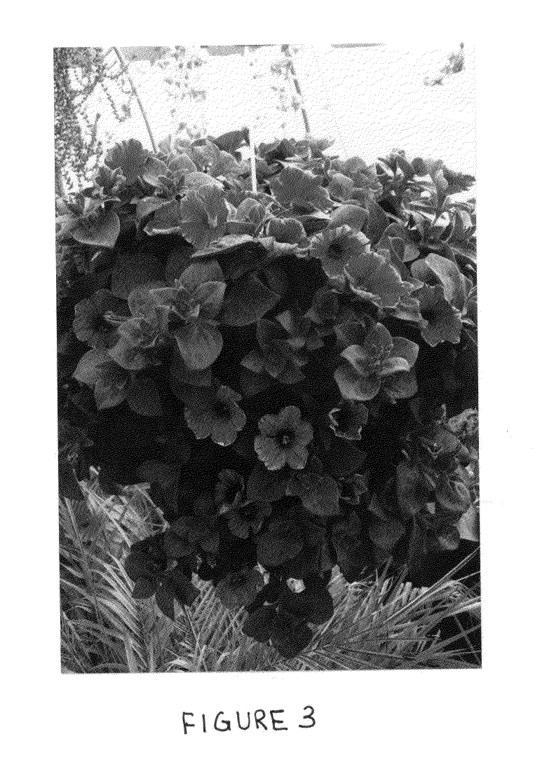

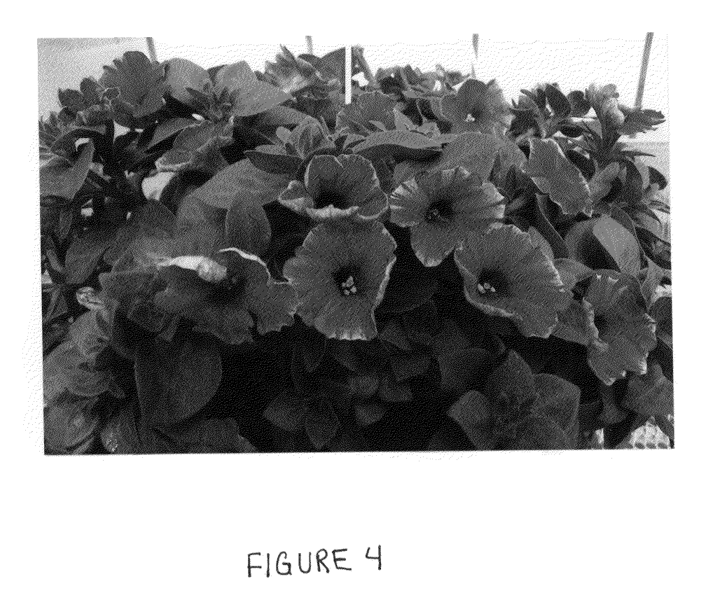

### DETAILED DESCRIPTION OF THE PREFERRED EMBODIMENT

The invention was discovered, reproduced and observed by the Applicant in Otisville, Mich. beginning in March, 2010. Asexual reproductions of the plant have been accomplished by vegetative cuttings since May 2010 have shown the unique features of the new plant to be stable and true to type in successive cuttings. The unique characteristics of the new plant include picotee type flowers with red to pink shades streaking throughout and a thin green edge and with thick dark green foliage. The plant is freely branching and includes an outwardly spreading/trailing habit.

The height of the plant is approximately 13 cm and flowers year around in moderate climate with a flower response which is indifferent to day length. The propagation is by vegetative cuttings with rooting by roots from the cutting base. Root initiation takes 7 to 10 days.

The foliage includes a leaves arranged alternatively shaped elliptic-simple with a leaf length of 6 cm for a mature leaf and a width of 3.8 cm of a pubescent texture. The color of the upper surface of the leaf is close to RHS143A. The stem is strong and freely branching with a length of 30 to 50 cm and a diameter of 0.5 to 0.8 cm with a pubescent texture and an internode length of 0.8 cm.

The flower is a single type facing upward to horizontal, funnel shaped with a flower bud length of 4 cm and a width of 1 cm. The color of the flower is a red to pink shade streaking from top of the throat to petal margin.

### SUMMARY OF THE PRESENT INVENTION

The following is a detailed description of the petunia plant, based upon my observations of the plant as grown in Otisville, Mich.
Flower type and habit: Single salverform flowers; flowers face outward and upward; freely flowering habit
Flowering season: Day length neutral; long flowering period; blooms year round in moderate climate
Flower size: Diameter: about 4.5 cm, Depth: about 4.5 cm, Tube length: about 2.7 cm
Flower buds: Length: about 4 cm. width: about 1 cm.
Petals: Quantity/arrangement: five petals fused in a single whorl, funnel form. Petal lobe length from throat: about 2 cm.
Petal lobe width: About 2 cm. Petal lobe margin: jagged, slightly upcurved. Petal lobe texture, upper surface: smooth with minute pubescence along margins. Petal lobe texture, lower surface: pubescent. Throat texture: smooth tube texture: pubescent Color: lateral red to pink shade color streaking from top of throat to petal margin, including colors close to rhs 44A, 49C, 50D, 54D, 55C; margin, close to 149A; venation, close to 44A; throat, close to 43A
Sepals: Single whorl of 5 sepals. Shape: subulate; Length: about 2.7 cm; Width: about 1 cm; Texture: pubescent; Color, upper surface: close to 141C; Color, lower surface: close to 141D. Peduncle. — Length: about 2 cm; Width: about 2 mm; Texture: pubescent; Color: close to 141D
Reproductive organs: Stamens: 5 per flower. filament length about 1.3 cm. Filament color white close to 157D. Anther shape oval, anther length about 2 mm, Pollen color close to 160C. Pistil: one per flower. Pistil length about 2 cm. Stigma shape oval; Stigma width about 3 mm Stigma color close to 142A. Style color close to 150B.
Seed/fruit: Seed and/or fruit production has not been observed.

It should be apparent to one with ordinary skill in the art that I have described a new and distinct variety of a petunia plant, DaSaJa1, characterized by dark green foliage and the color of the flower with red to pink shades streaking throughout and a thin green edge.

Having now described the new and distinct variety of petunia plant which I have discovered and asexually produced,

I claim:

1. A new and distinct variety of petunia plant, substantially as herein illustrated and described, characterized by its distinctive flower colors and its dark green foliage
